(19) United States
(12) Patent Application Publication
(10) Pub. No.: US 2022/0401595 A1
(43) Pub. Date: Dec. 22, 2022

(54) SANITIZING BOOTH

(71) Applicant: SaniPort, LLC, Naples, FL (US)

(72) Inventors: Stephen LaMelza, Naples, FL (US); Gregory Fowler, Naples, FL (US)

(21) Appl. No.: 17/745,390

(22) Filed: May 16, 2022

Related U.S. Application Data
(60) Provisional application No. 63/212,859, filed on Jun. 21, 2021.

(51) Int. Cl. A61L 2/00; A61L 2/26

(52) U.S. Cl. CPC A61L 2/0088; A61L 2/26; A61L 2202/15; A61L 2101/06

(57) ABSTRACT

A sanitizing booth includes integrally formed upper and lower body structures attached to one another to collectively define a compartment interior space. The lower body structure has side walls collectively defining an enclosed reservoir cavity for receiving a sanitizing solution therein. A first conduit extends from the ceiling of the upper body structure into the reservoir cavity. At least one second conduit extends downward from the ceiling along the interior surface of the compartment body. At least one sprayer is attached to the at least one second conduit for spraying the sanitizing solution. A pump arrangement in fluid communication with the first conduit and the at least one second conduit is configured to selectively and controllably draw the sanitizing solution from the reservoir cavity through the first conduit and pass it through the at least one second conduit and the at least one sprayer into the compartment interior space.

### CROSS-REFERENCE TO RELATED APPLICATION

The present application claims priority to U.S. Provisional Application 63/212859 filed on Jun. 21, 2021 and entitled “Sanitizing Booth”, the complete disclosure of which is incorporated herein by reference.

### TECHNICAL FIELD

This application relates generally to sanitizing systems and, more particularly, to a self-contained walk-through booth or portal system for applying a disinfectant or sanitizing mist to subjects.

### BACKGROUND

Recent events have prompted a flurry of development in technology aimed at mitigating risks associated with pathogens that may be inadvertently transferred from surface-to-surface. Of particular concern is the presence of such pathogens on the clothing and skin of individuals entering a highly occupied enclosed space. Accordingly, a number of mechanisms for sanitizing such individuals are being developed for use in airports, terminals, arenas, etc.

There exist deficiencies with these existing sanitizing mechanisms. Accordingly, there is a need for a self-contained walk-through booth or portal system for applying a disinfectant or sanitizing mist to subjects that overcomes these deficiencies.

### SUMMARY

An illustrative aspect of the present application provides a sanitizing booth comprising an integrally formed upper body structure and an integrally formed lower body structure. The upper body structure has a pair of opposing upper body side walls and an upper ceiling. The lower body structure has a pair of opposing lower body side members and an integral floor structure. At least a portion of the lower body structure comprises opposing thin side walls collectively defining an enclosed reservoir cavity adapted for receiving a sanitizing solution therein. The lower body structure is attached to the upper body structure to form a compartment body having an interior surface defining a compartment interior space. The compartment interior space has opposing compartment side members and front and rear openings sized for entry into and exit from the interior space by a subject. The sanitizing booth further comprises a first conduit disposed within the compartment body, extending from the upper ceiling through the upper body structure into the reservoir cavity of the lower body structure. The sanitizing booth also comprises at least one second conduit extending downward from the upper ceiling along the interior surface of the compartment body and at least one sprayer operatively attached to the at least one second conduit for receiving the sanitizing solution and spraying the sanitizing solution into the compartment interior space. An equipment and control module is attached to the upper ceiling of the upper body. The equipment and control module comprises a pump arrangement and a pump control circuit. The pump arrangement is in fluid communication with the first conduit and the at least one second conduit and is sized and configured to selectively and controllably draw the sanitizing solution from the reservoir cavity through the first conduit and pass the sanitizing solution into and through the at least one second conduit and through the at least one sprayer into the compartment interior space.

Further features of the disclosed sanitizing booths, and the advantages offered thereby, are explained in greater detail hereinafter with reference to specific example embodiments illustrated in the accompanying drawings.

### BRIEF DESCRIPTION OF THE DRAWINGS

The invention can be more fully understood by reading the following detailed description together with the accompanying drawings, in which like reference indicators are used to designate like elements. Note that the illustrated embodiments are provided as examples of the present invention. They should not be considered as limited factors to the design and shape of the invention, but as examples.

FIG. 1 is a perspective rendering of a sanitization booth according to an embodiment of the invention;

FIG. 2 is a perspective rendering of a sanitization booth according to an embodiment of the invention;

FIG. 3 is a front view of a sanitization booth according to an embodiment of the invention;

FIG. 4 is a top view of the lower body structure of the sanitization booth of FIG. 3;

FIG. 5 is a side view of the sanitization booth of FIG. 3;

FIG. 6 is a section view of the sanitization booth of FIG. 3;

FIG. 7 is a sight glass portion of the sanitization booth of FIG. 3;

FIG. 8 is a perspective view of an equipment and control module of a sanitization booth according to an embodiment of the invention;

FIG. 9 is a front exploded view of the equipment and control module of FIG. 8;

FIG. 10 is a schematic view of a control board and a liquid pumping and distribution system for usable in embodiments of the invention;

FIG. 11 is a schematic view of a control board usable in embodiments of the invention; and

FIG. 12 is a schematic view of a liquid delivery system usable in embodiments of the invention.

### DETAILED DESCRIPTION OF THE INVENTION

While the invention will be described in connection with particular embodiments, it will be understood that the invention is not limited to these embodiments. On the contrary, it is contemplated that various alternatives, modifications and equivalents are included within the spirit and scope of the invention as described. In the description of the invention, the majority of references are to an example used as a surface mount. If otherwise, it will be stated.

The present invention provides an automatic or semi-automatic sanitizing booth system for disinfecting and/or sanitizing a subject. As used herein, a subject can be an object or item (e.g., a backpack bag, a suitcase, a piece of clothing, and the like), an animal (e.g., a cat, a pet, a dog, and the like), or an individual human being. The system may comprise a tunnel-like, step-in booth having an arrangement of misters or sprayers and an integral reservoir for disinfectant/sanitizing solution. When a subject enters the booth, the system may be selectively or automatically activated to spray disinfectant/sanitizing solution through multiple nozzles towards the center of the booth where the subject stands. Spraying may automatically stop after a preset period of time (adjustable). When the nozzles stop spraying, the subject can exit from an end opposite to the end from which the subject is entered. The system may have an adjustable dwell time before the activation switch can be pressed again (adjustable). The system may be capable of sanitizing approximately 400 to 1,500 subjects/people per hour depending on machine setup specifications of the system.

To facilitate description, Table 1 lists the reference numbers of a sanitizing booth system and its components disclosed herein.

TABLE 1
Sanitizing Booth System/Components

| Ref. No | System/Component |
|---|---|
| 100 | Sanitizing booth system |
| 110 | Upper body structure |
| 111 | Upper body thin wall |
| 112 | Conduit port |
| 113 | Upper body cavity |
| 114 | Top support platform |
| 119 | Upper tube trough |
| 120 | Lower body structure |
| 121 | Lower body thin wall |
| 123 | Lower body cavity/reservoir |
| 124 | Integral support leg |
| 125 | Body structure base |
| 126 | Lower body side wall recess |
| 127 | Fastening surface (for positioning fastener(s) |
| | to connect upper and lower structures) |
| 129 | Lower tube trough |
| 130 | Equipment and control module |
| 131 | Top cover |
| 132 | Equipment chassis |
| 134 | Pump/accumulator arrangement |
| 136 | Control circuitry |
| 138 | Air screen |
| 139 | LED |
| 140 | Subject support platform |
| 150 | Reservoir filler port and cover |
| 152 | Reservoir level determination arrangement |
| 154 | Magnifying sight glass |
| 160 | Activation button |
| 170 | Liquid delivery arrangement |
| 171 | Delivery tube |
| 172 | Nozzle |
| 180 | Joint guide structure |
| 190 | Joint guide structure |

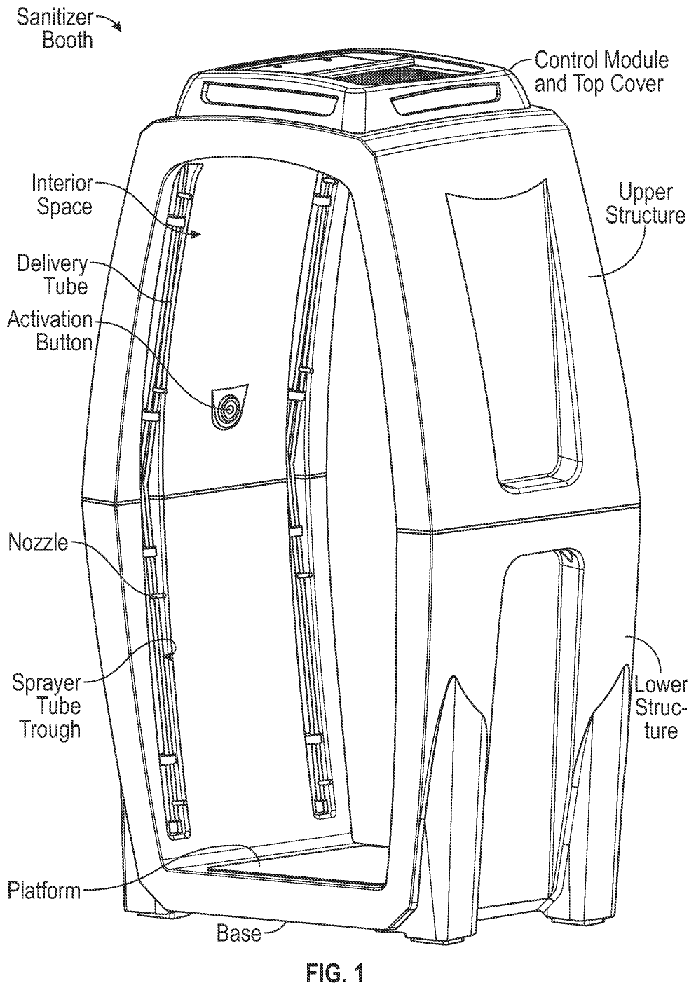

FIG. 1 illustrates an exemplary sanitizing booth system according to an embodiment of the invention. The sanitizing booth may comprise a main body structure having an integrally formed upper portion (an integrally formed upper body structure) and an integrally formed lower portion (an integrally formed lower body structure). The upper and lower structures may be joined together (e.g., by bolting or other fastening means) to collectively define an interior space into which a subject steps to undergo the sanitizing process. For example, the lower body structure is attached to the upper body structure to form a compartment body. The compartment body has an interior surface defining a compartment interior space having opposing compartment side members and front and rear openings sized for entry into and exit from the interior space by a subject. The lower structure is provided with lower side walls and an integral base that supports a platform for supporting the subject within the compartment interior space. For example, the lower structure has a pair of opposing lower body side members and an integral floor structure. At least a portion of the lower body structure comprises opposing thin side walls collectively defining an enclosed reservoir cavity adapted for receiving a sanitizing solution therein. The upper structure is provided with a pair of opposing upper body side walls and an upper ceiling portion that supports a control module, operating equipment (pumps, accumulator tank, fans, solenoids, etc.), and a top cover. In this exemplary embodiment, both the upper and lower structures each have two sprayer tube troughs on the interior-facing surface of each side wall. These troughs are configured to receive delivery tubes (sometimes referred to as sprayer tubes) configured for transferring sanitizing solution from the reservoir in the lower structure to a plurality of spray or misting nozzles.

The upper and lower body structures may each be formed as a single integral structure having thin walls defining a corresponding enclosed cavity. Some or all of the enclosed cavity in the lower structure may be used as the reservoir for a sanitizing solution. The upper and lower structures may be formed from any of various moldable thermoplastic and/or composite materials. These may include, but are not limited to, low density polyethylene (LDPE), medium density polyethylene (MDPE), high density polyethylene (HDPE), fiberglass, acrylonitrile butadiene styrene (ABS), and nylon. In particular embodiments, the upper and lower structures may be molded LDPE, MDPE, HDPE or a polyethylene blend. In some embodiments, the upper and lower structures may be 3D-printed.

The upper body structure provides a support platform for the control module and an equipment chassis or tray on which some or all of the components of a pumping arrangement configured for drawing liquid from the reservoir and transferring it through the delivery tubes to the nozzles under sufficient pressure to spray or atomize and distribute the liquid through the booth. In particular embodiments, the pumping arrangement may include a low pressure pump and an accumulator tank from which liquid may be selectively drawn to a high pressure pump for distribution to the pipes/delivery tubes via manifold.

The sanitizing liquid may be any suitable liquid that can be stored safely in the internal reservoir drawn through the pumping and distribution components and sprayed into the booth through the nozzles. One exemplary liquid that may be suitable would be a hypochlorous acid solution. The lower body structure is formed from a moldable material selected for compatibility with the sanitizing solution/liquid.

The control module may include control and power circuitry for controlling the actions of the pumps. In some embodiments, the control and power circuitry may be configured for receiving power from a standard external 115 VAC input line. In other embodiments, the booth system may be powered by an on-board battery. In some embodiments, the control module may include one or more data processors and/or timers. These may be programmed or otherwise configured so that activation of the booth system results in a controlled misting cycle with a predetermined duration. In some embodiments, they may also be programmed so that there is a predetermined minimum time delay between activations. The control circuitry may be in electrical communication with an activation mechanism (e.g., a button or other device) mounted in the interior space. In some embodiments, light curtains, pressure sensors, or other mechanism for sensing subject entry may be used for automatic activation. In some embodiments, the control circuitry may be in electrical communication with one or more sensors in the liquid reservoir configured for providing information on the amount of liquid in the reservoir. In some embodiments, the liquid level in the reservoir may be determined through the use of sight windows installed through the lower structure wall at strategically placed locations.

In some embodiments of the booth system may include one or more light emitting diode (LED) lights conspicuously mounted to the top cover or main body of the booth system. These lights may be configured as indicators of whether the booth is ready for use. For example, a green LED indicator may indicate that the system is powered and ready for use. A red indicator may indicate that the system is in use or is not yet ready for use. The activation button may also include one or more such indicator lights.

In some embodiments, the sanitizing booth may comprise a first conduit disposed within the compartment body extending from the upper ceiling through the upper body structure into the reservoir cavity of the lower body structure. The sanitizing booth may further comprise at least one second conduit extending downward from the upper ceiling along the interior surface of the compartment body. At least one sprayer is operatively attached to the at least one second conduit for receiving the sanitizing solution and spraying the sanitizing solution into the compartment interior space. The equipment and control module is attached to the upper ceiling of the upper body. The equipment and control module may comprise a pump arrangement and a pump control circuit. The pump arrangement is in fluid communication with the first conduit and the at least one second conduit and is sized and configured to selectively and controllably draw the sanitizing solution from the reservoir cavity through the first conduit and pass the sanitizing solution into and through the at least one second conduit and through the at least one sprayer into the compartment interior space. The first conduit may be integrally formed in the compartment body, for example, inside the upper and lower body structures. The first conduit may also be formed of a metal or plastic tube attached to the compartment body. The at least one second conduit may be formed of one or more metal or plastic tubes. The at least one sprayer includes a plurality of sprayers collectively arranged and disposed with the at least one second conduit so as to distribute the sanitizing solution around the subject positioned within the compartment interior space. Further, the interior surface comprises at least one trough opening into the compartment interior space, and each of the at least one trough receives one of the at least one second conduit disposed therein.

In some embodiments, the sanitizing booth may comprise one or more cameras, light sensors, or pressure sensors may be installed in the compartment interior space. Any or all of these may be monitored and controlled by control circuitry in the equipment and control module, the foregoing being collectively configured to detect a presence of a subject inside the compartment interior space. The pump control circuit is configured to initiate a sanitation spray cycle upon wherein detecting the presence of a subject inside the compartment interior space.

In some embodiments, the activation button is mounted on the interior surface, and is in electrical communication with the pump control circuit and configured for activation of a sanitation spray cycle.

In some embodiments, the sanitizing booth may comprise one or more fluid level sensors arranged inside the reservoir cavity. The one or more fluid level sensors are in electrical communication with the pump control circuit.

In some embodiments, the lower body structure is provided with one or more sight glass windows through one of the thin walls defining the reservoir cavity. The one or more sight glass windows are positioned to provide a view of a liquid level of the sanitizing solution disposed within the reservoir cavity.

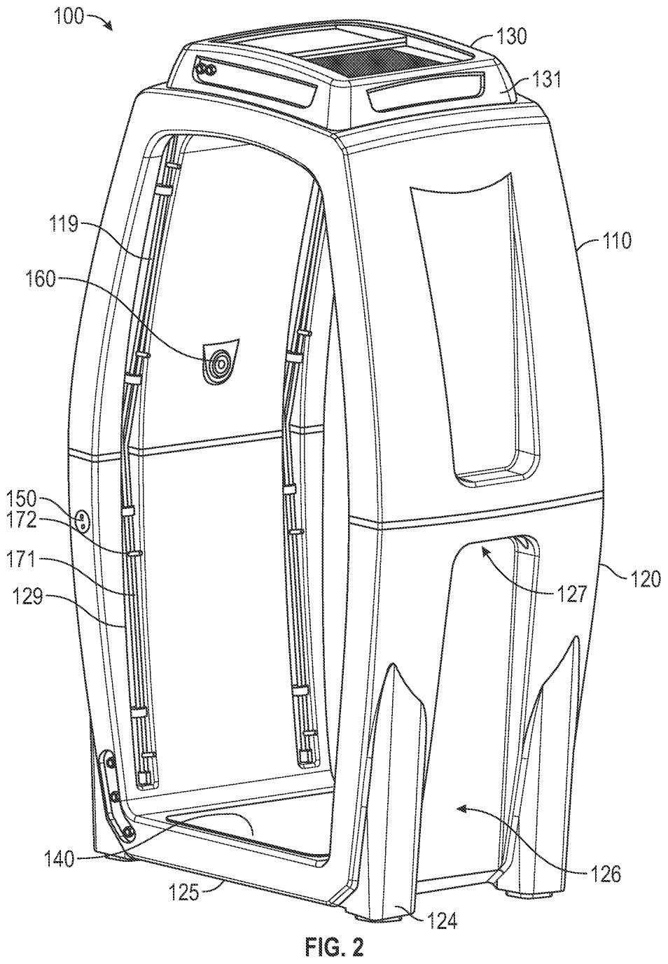

FIG. 2 is a perspective rendering of a sanitization booth system 100 according to an embodiment of the invention. The system 100 may comprise an upper body structure 110 and a lower body structure 120. The lower body structure 120 may be fixedly or removably coupled to the upper body structure 110 to collectively define an interior space into which a subject is entered for a sanitizing process. The system 100 may further comprise an equipment and control module 130 fixedly or removably coupled to a top end surface of the upper body structure 110.

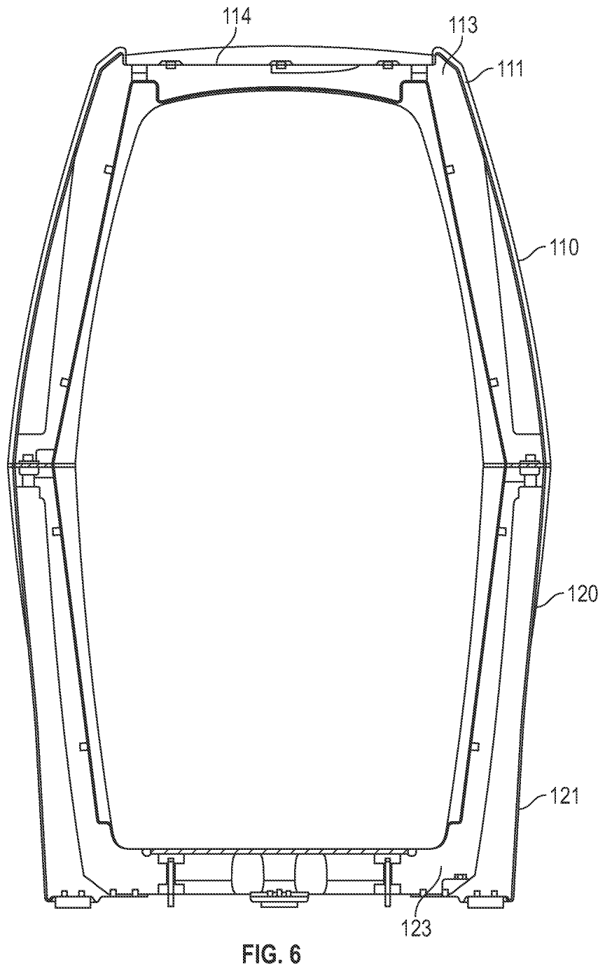

As shown in FIG. 6, the upper body structure 110 may be formed with double thin side walls 111 on each side portion of the upper body structure 110 to define a corresponding enclosed upper body cavity 113. The upper body structure 110 may be provided with a top support platform 114.

Referring back to FIG. 2, the upper body structure 110 may be provided with one or more upper tube troughs 119 for receiving a corresponding number of delivery tubes 171. Each delivery tube 171 runs through a corresponding upper tube trough 119 for delivering the sanitization solution to the nozzles 172 in the upper structure. The nozzles 172 are configured to form one or more corresponding sprayers for spraying the sanitization solution onto the subject when needed. Specifically, a sprayer may comprise one or more nozzles.

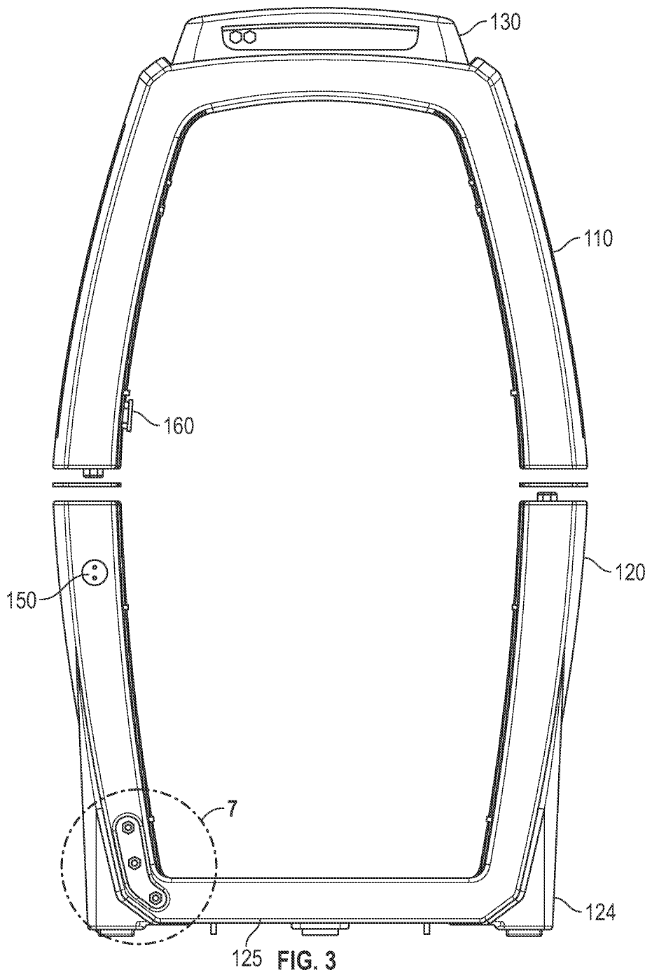

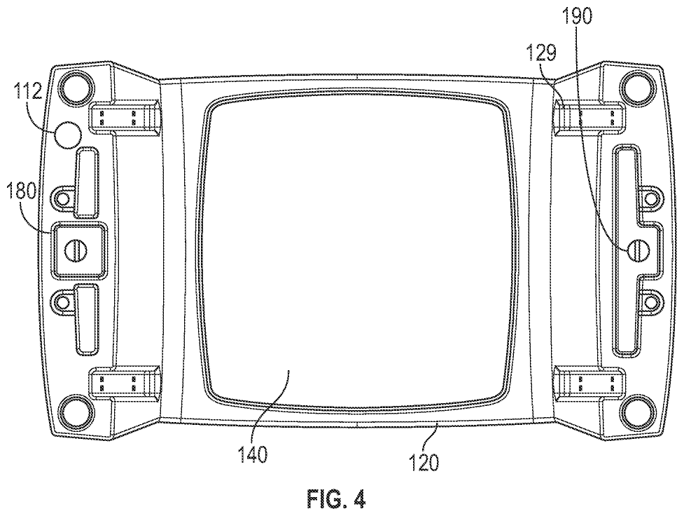

The lower body structure 120 can be bolted to, threaded to, or hinged to the upper body structure 110 as illustrated in FIG. 3. In some embodiments, the lower body structure 120 may be permanently bonded to the upper body structure 110. As shown in FIG. 4, a top view of the lower body structure 120 of the sanitization booth system 100 is illustrated to show some mechanisms/features 180 and 190 that can be configured for joining the lower body structure 120 with the upper body structure 110. Also shown in FIG. 2 and FIG. 4, the lower body structure 120 may be provided with a subject support platform 140 for supporting a subject on which the subject can stand, sit, or be placed.

As shown in FIG. 6, the lower body structure 120 may also be formed with double thin side walls 121 on each side portion of the lower body structure 120 to define an enclosed reservoir cavity 123 for receiving and storing a sanitizing solution.

Referring to FIG. 2, the lower body structure 120 may be formed with a body structure base 125 on which the subject support platform 140 is arranged. The lower body structure 120 may be provided with one or more lower tube troughs 129 which may be configured to align with the upper tube troughs 119 for receiving the delivery tubes 171 for delivering the sanitization solution to the nozzles 172 in the lower portion of the structure.

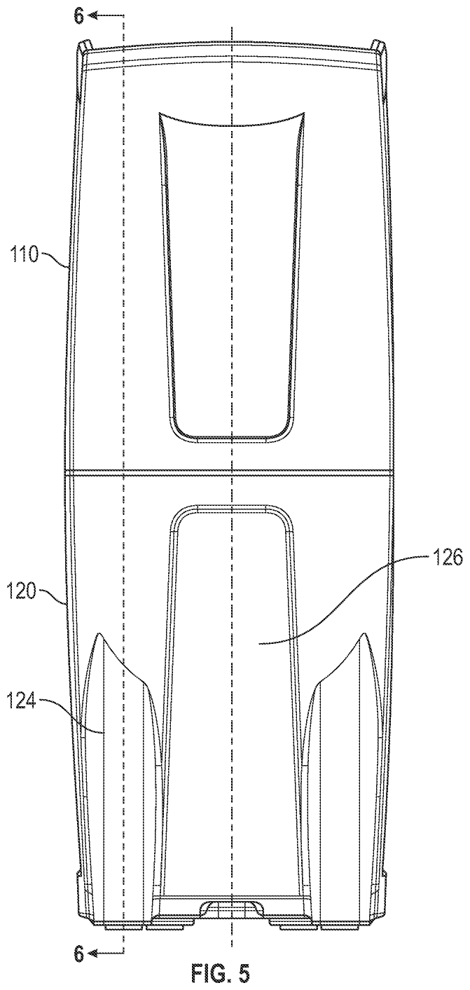

Referring to FIG. 2, FIG. 3 and FIG. 5, the lower body structure 120 may be formed with integral support legs 124. Each of the integral support legs 124 is formed in a corresponding portion of the lower body structure 120. The lower body structure 120 may also be formed with two lower body side wall recesses 126, each of which is located on a corresponding side of the lower body structure 120 facing away the interior space.

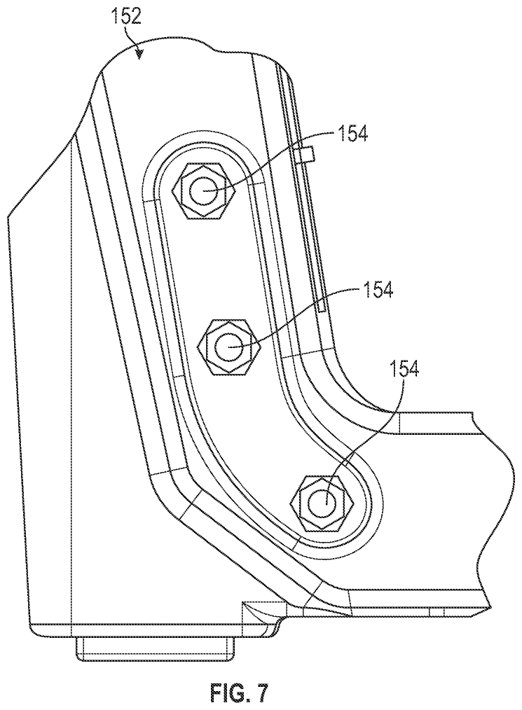

As shown in FIG. 2 and FIG. 3, a reservoir filler port and cover 150 may be provided at an upper portion of one side of the lower body structure 120. The reservoir filler port and cover 150 may be used for filling the sanitization solution into the reservoir cavity 123. The reservoir cavity 123 may further be provided with a reservoir level determination arrangement 152 at a lower portion of one side of the lower body structure 120. Referring to FIG. 7, the reservoir level determination arrangement 152 may include a sight glass portion comprising one or more magnifying sight glass windows 154, which is used for viewing, monitoring, or determining a liquid level of the sanitization solution stored in the reservoir cavity 123.

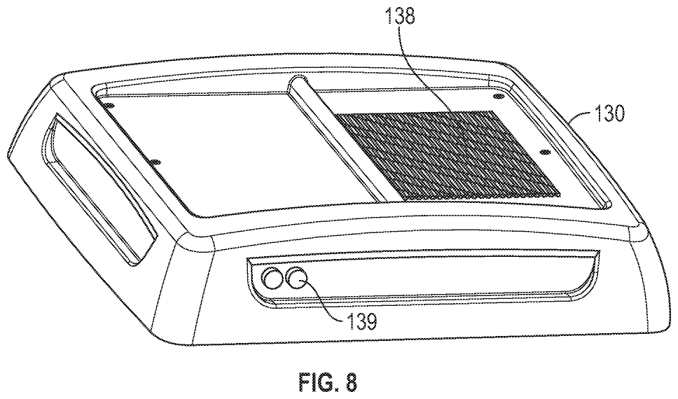

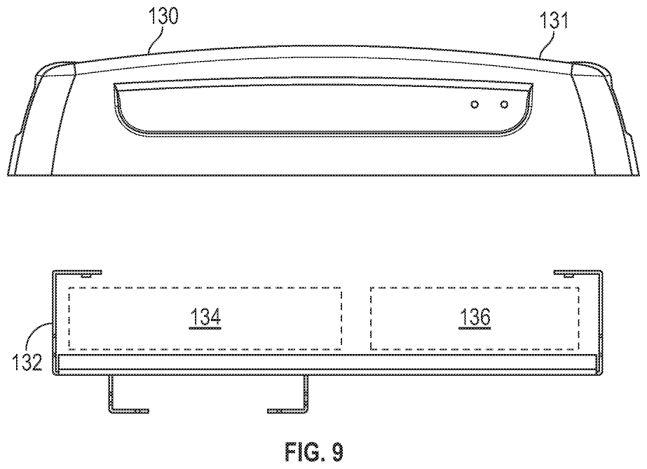

Referring to FIG. 2, FIG. 8 and FIG. 9, the equipment and control module 130 may comprise a top cover 131 and an equipment chassis or tray 132. The equipment chassis or tray 132 is configured to receive a pump/accumulator arrangement 134 and a control circuitry 136. The pump/accumulator arrangement 134 may comprise a lower pressure pump, an accumulator tank, and a high pressure pump. The input side of the pump/accumulator arrangement 134 may be connected to a first conduit 133 that provides fluid communication between the reservoir 123 and the pump arrangement 134. The output side may be connected to a second conduit 135 that connects to or is part of a liquid delivery arrangement 170 that includes the delivery tubes 171 and the nozzles 172. The first conduit 133 may be or include a tube arranged to pass through the upper body cavity 113 and through a port 112 into the reservoir cavity 123 in the lower body structure 120. In some embodiments, some or all of the first conduit 133 may be a tube formed as an integral part of the upper body structure 110. In either case, the first conduit 133 is connected to the input side of the low pressure pump for drawing liquid from the reservoir cavity 123 into the accumulator tank. The high pressure pump is configured to transfer the sanitization solution from the accumulator tank to the second conduit 135, which may be connected to or a part of the liquid delivery arrangement 170.

As described above, the control circuitry 136 is configured to control functions of various components of the system 100, such as the high and low pressure pumps, the LED lights, the sensors, the solenoids, etc. The equipment and control module 130 may further comprise an air screen 138 for air circulation and/or protection of the components inside the equipment and control module 130. For example, the air screen 138 may be used for air circulation for cooling down the low and high pressure pumps, and may also be used for maintenance of the inside components. The air screen 138 may be disposed under the top cover 131. As shown in FIG. 8, the equipment and control module 130 may further comprise one or more LED lights 139 at a suitable position of the equipment and control module 130 (on a frond side of the equipment and control module 130 in this example).

Referring to FIGS. 2 and 3, the system may further comprise an activation button 160. The activation button 160 is in an electrical communication with the control circuitry 136, and controlled by the control circuitry 136 for activating/deactivating or stopping the system 100. For example, the activation button 160 can be pressed to activate the nozzles to spray liquid onto a subject, and can be controlled by the control circuitry 136 to deactivate the nozzles after a predetermined period of time.

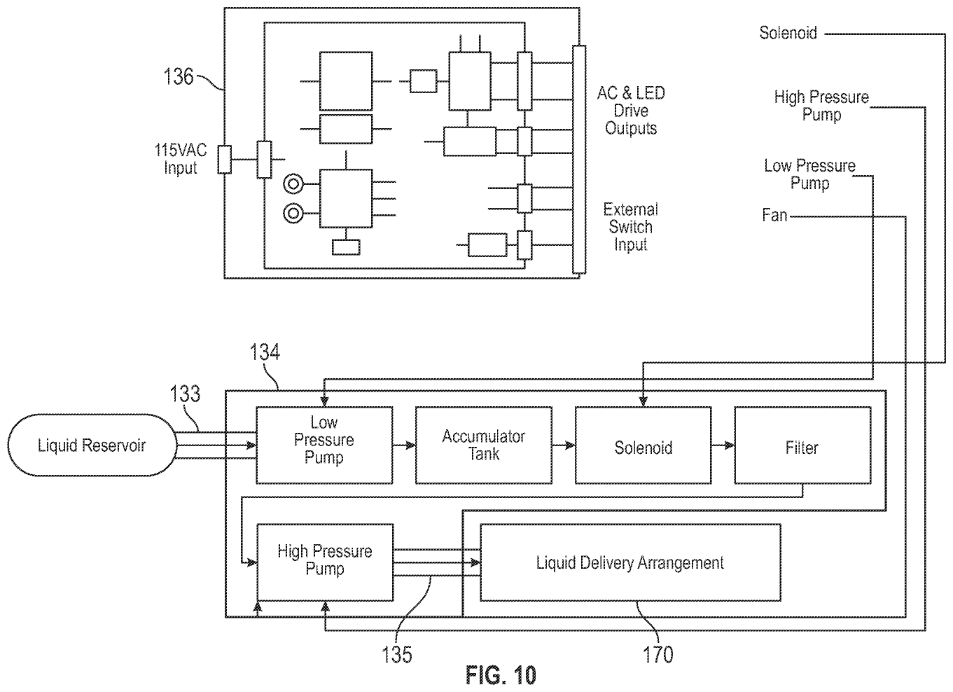

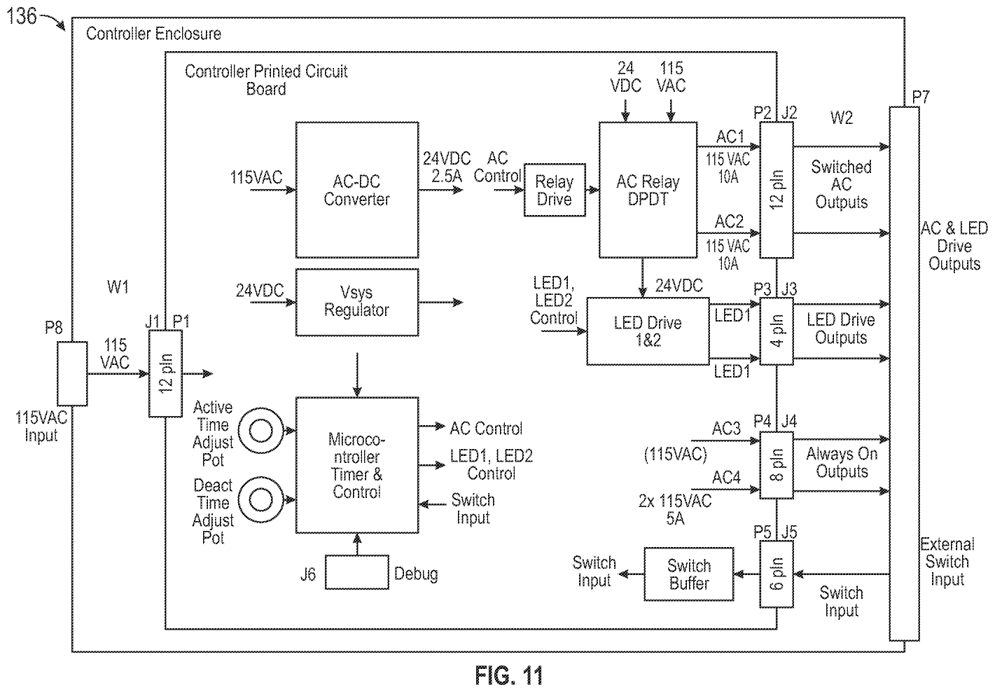

FIGS. 10 and 11 illustrate a schematic diagram view of an example control circuitry 136 and a schematic diagram of an example liquid pumping arrangement 134 and a liquid distribution/delivery arrangement 170, which can be implemented in the invention. The liquid pumping arrangement 134 may further include one or more fans, one or more solenoids, and one or more filters. The one or more fans can be configured to dissipate heat generated inside the equipment and control module 130. The one or more solenoids can be configured to switch on, switch off, and/or regulate the liquid flow of the sanitization solution. The one or more filters can be used for filtering the sanitization solution prior to distribution by the high pressure pump to the liquid delivery arrangement 170.

The control circuitry 136 may comprise various components such as power supply, timer, LED driver, solenoid control, pump controls, and input circuits. The control circuitry 136 is configured to control the one or more solenoids, the one or more fans, the high pressure pump, the low pressure pump, etc. The sanitization liquid stored in the liquid reservoir 123 is drawn by the low pressure pump into the accumulator tank, and then delivered from the accumulator tank by the high pressure pump to the liquid delivery arrangement 170 through the solenoid and filter.

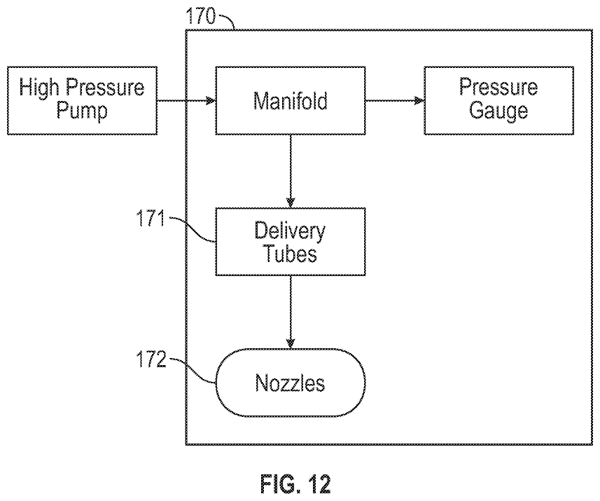

A schematic diagram view of an example liquid delivery arrangement 170 is illustrated in FIG. 12, which can be implemented in the invention. The example liquid delivery arrangement 170 may comprise a manifold, a pressure gauge, the delivery tubes 171, and the nozzles 172. The manifold may be configured to regulate the liquid flow of the sanitization solution pumped from the accumulator tank by the high pressure pump. The pressure gauge is configured to measure, monitor, or regulate the liquid pressure of the sanitization solution. The pressure gauge may be any type of suitable pressure gauges, such as a Bourdon pressure gauge, membrane pressure gauge, pneumatic pressure regulator, and the like.

In some embodiments, at least one sprayer comprising one or more nozzles may be disposed on the upper body structure 110 and/or the lower body structure 120 facing the interior space and configured to spray the sanitizing solution onto the subject.

In some embodiments, the equipment and control module 130 may be arranged atop the upper body structure 110 and coupled fixedly and removably to the integral upper ceiling (where the top support platform 114 is situated) of the upper body structure 110. The equipment and control module 130 is configured to fluidly communicate with the at least one sprayer and the enclosed reservoir cavity 123 in which the sanitizing solution is stored, for distributing the sanitizing solution.

The top cover 131 of the equipment and control module 130 may be coupled to the equipment tray 132 to collectively define an enclosed chamber. The lower pressure pump is fluidly coupled to the liquid accumulator, and the high pressure pump is fluidly coupled to the liquid accumulator. The liquid accumulator tank, the lower pressure pump, and the high pressure pump are all arranged inside the enclosed chamber. The control circuitry board 136 is arranged inside the enclosed chamber and configured to control the at least one sprayer/nozzles, the lower pressure pump, and the high pressure pump.

As previously described, the upper and lower body structures 110, 120 may each include at least one tube trough 119, 129 formed on an inward-facing surface thereof. The upper trough(s) 119 and lower trough(s) 129 may be arranged to align with one another and two receive a sprayer/delivery tube 171 therein.

An activation button 160 may also be mounted to an inward facing wall in the interior space. The activation button may be wired for electrical communication with the equipment and control module 130 for selectively activating a sanitization cycle of the system.

In some embodiments, at least one light emitting diode (LED) light 139 may be mounted on one or more of the upper body structure 110, the lower body structure 120, and the equipment and control module 130. The at least one LED light 139 may be configured as an indicator indicating an operation status of the sanitizing booth system, such as a status of “in use,” a status of “ready,” a status of “in maintenance,” or the like.

In some embodiments, one or more sensors may be arranged inside the reservoir cavity 123 for providing information on an amount of the sanitizing solution inside the reservoir cavity 123. The sensors may be any type of suitable sensors, such as optical sensors, pressure sensors, and the like. The sensors are in signal communication with the control circuitry board 136. In addition or as an alternative, the lower body structure 120 may be provided with one or more sight glass windows 154 positioned so as to provide a view inside the reservoir cavity 123. These sight glass windows 154 may be configured for determining a liquid level of the sanitizing solution inside the reservoir cavity 123. The lower body structure 120 may also be provided with a liquid filling port and cover 150 positioned near an upper portion of the reservoir cavity 123.

In some embodiments, the sanitizing booth system disclosed herein may further comprise one or more cameras installed in the interior space. The one or more cameras are controlled by the control circuitry 136 in the equipment and control module 130, and are configured to detect presence of a subject inside the interior space. For example, the cameras working with corresponding computer software may be used for image recognition (e.g., object identification, facial recognition) to determine a type of the subject (e.g., a human being, an animal). Accordingly, different subjects may receive different amounts of sanitation solution sprayed and/or receive different spraying durations. Further, different portions of a subject can receive different amounts of sanitation solution sprayed and/or receive different spraying durations. For example, the face of a person may be avoided for spraying liquid, the upper body of the person may be sprayed more liquid or a longer duration, and the lower body of the person may be sprayed less liquid or a shorter duration. The sprayers/nozzles can be controlled by the control circuitry 136 to orientate to spray the sanitation solution for different heights and/or different orientations.

In some embodiments, the sanitizing booth system disclosed herein may be operated in a complete automatic mode. For example, when the one or more cameras detect the presence of a subject inside the interior space, the control circuitry 136 in the equipment and control module 130 can be configured to automatically activate the at least one sprayer/nozzles to spray the sanitizing solution onto the subject, without pressing the activation button 160 by the subject.

A typical use scenario for a sanitizing booth system disclosed herein similar to that of FIG. 1 may include the following actions:
1. Ensure the sanitizing system has a minimum amount of sanitation/disinfectant fluid for operation.
2. Connect the sanitizing system to a power source.
3. Unit will power up and green LEDs will illuminate on the switch and on the top cover.
   a. The low-pressure pump will start filling the accumulator tank. It will run until a low-pressure switch activates.
4. Entry of the unit is defined by the LED lights on the top cover and will be visible at entry, as well as the green LED ring switch (activation button) inside the unit as a user enters (right side to the user).
5. Once inside booth and standing in the middle of the unit, the subject activates the system by touching/pushing the activation button.
   a. When the button is touched/pushed, it starts a preset timed relay. One relay activates a solenoid to allow low pressure water from the accumulator tank to pass through an inline filter and into the high-pressure pump. At the same time, the second relay starts the high-pressure pump that produces the mist inside the booth interior with the delivery tubes and misting nozzles.
6. Upon activation, the LEDs will change from green to red (red indicates “cycling”). The green LED on the top cover will turn off and the red LED will illuminate while the mist cycle runs.
   a. Once the preset time period has expired the relays return to the at rest state and the solenoid stops the low-pressure water flow to the inline filter and high-pressure pump. The misting then stops. The cycle may include a preset period of post-mist dwell time.
7. When the mist cycle ends, the switch (activation button) LED ring will return to green and the green LED in the top cover will again illuminate. Both red LEDs will turn off
   a. The low-pressure pump will restart only when the internal preset pressure switch allows it.
8. When the green LED lights are on, the user may then exit the booth by stepping out in the opposite opening that the user walked in.

It will be understood that the exact configuration and dimensions provided herein are exemplary only and the invention is not limited thereby. Table 1 indicates the components identified by the reference numbers shown in FIGS. 2-11.

It will be readily understood by those persons skilled in the art that the present invention is susceptible to broad utility and application. Many embodiments and adaptations of the present invention other than those herein described, as well as many variations, modifications and equivalent arrangements, will be apparent from or reasonably suggested by the present invention and foregoing description thereof, without departing from the substance or scope of the invention.

What is claimed is:

1. A sanitizing booth, comprising:
an integrally formed upper body structure having a pair of opposing upper body side walls and an upper ceiling;
an integrally formed lower body structure having a pair of opposing lower body side members and an integral floor structure, at least a portion of the lower body structure comprising opposing thin side walls collectively defining an enclosed reservoir cavity adapted for receiving a sanitizing solution therein, the lower body structure being attached to the upper body structure to form a compartment body having an interior surface defining a compartment interior space having opposing compartment side members and front and rear openings sized for entry into and exit from the interior space by a subject;
a first conduit disposed within the compartment body, extending from the upper ceiling through the upper body structure into the reservoir cavity of the lower body structure;
at least one second conduit extending downward from the upper ceiling along the interior surface of the compartment body;
at least one sprayer operatively attached to the at least one second conduit for receiving the sanitizing solution and spraying the sanitizing solution into the compartment interior space; and
an equipment and control module attached to the upper ceiling of the upper body, the equipment and control module comprising a pump arrangement and a pump control circuit, the pump arrangement being in fluid communication with the first conduit and the at least one second conduit and being sized and configured to selectively and controllably draw the sanitizing solution from the reservoir cavity through the first conduit and pass the sanitizing solution into and through the at least one second conduit and through the at least one sprayer into the compartment interior space.

2. The sanitizing booth of claim 1, wherein the lower body structure includes an integral base that supports a platform for supporting the subject within the compartment interior space.

3. The sanitizing booth of claim 1, wherein the pump arrangement comprises a liquid accumulator tank, a lower pressure pump fluidly coupled to the liquid accumulator, and a high pressure pump fluidly coupled to the liquid accumulator.

4. The sanitizing booth of claim 1, wherein the at least one sprayer is a plurality of sprayers collectively arranged and disposed with the at least one second conduit so as to distribute the sanitizing solution around the subject positioned within the compartment interior space.

5. The sanitizing booth of claim 1, wherein each of the at least one sprayer includes one or more spraying nozzles.

6. The sanitizing booth of claim 1, wherein the sanitizing solution is a hypochlorous acid solution.

7. The sanitizing booth of claim 1, wherein the lower body structure is formed from a moldable material selected for compatibility with the sanitizing solution.

8. The sanitizing booth of claim 1, wherein the upper and lower body structures are each formed from a moldable material comprising at least one of the set consisting of a thermoplastic material and a composite material.

9. The sanitizing booth of claim 1, wherein the interior surface comprises at least one trough opening into the compartment interior space, each of the at least one trough having one of the at least one second conduit at least partially disposed therein.

10. The sanitizing booth of claim 1, further comprising an activation button mounted on the interior surface, the activation button being in electrical communication with the pump control circuit and configured for activation of a sanitation spray cycle.

11. The sanitizing booth of claim 1, further comprising at least one light emitting diode (LED) light mounted on one or more of the upper body structure, the lower body structure, and the equipment and control module, the at least one LED light configured as an indicator indicating an operation status of the sanitizing booth.

12. The sanitizing booth of claim 1, further comprising one or more fluid level sensors arranged inside the reservoir cavity, the one or more fluid level sensors being in electrical communication with the pump control circuit.

13. The sanitizing booth of claim 1, wherein the lower body structure is provided with a liquid filling port arranged in an upper portion of the reservoir cavity.

14. The sanitizing booth of claim 1, wherein the lower body structure is provided with one or more sight glass windows through one of the thin walls defining the reservoir cavity, the one or more sight glass windows being positioned to provide a view of a liquid level of the sanitizing solution disposed within the reservoir cavity.

15. The sanitizing booth of claim 1, further comprising one or more cameras installed in the compartment interior space, the one or more cameras controlled by a control circuitry in the equipment and control module, and configured to detect a presence of the subject inside the compartment interior space.

16. The sanitizing booth of claim 15, wherein the pump control circuit is configured to initiate a sanitation spray cycle upon detecting the presence of the subject inside the compartment interior space.

17. The sanitizing booth of claim 1, wherein at least a portion of the first conduit is an integrally formed part of the upper body structure.

18. The sanitizing booth of claim 1 wherein the first conduit comprises a flexible tube at least partially disposed within an upper body structure cavity of one of the upper body side walls.
